(19) United States
(12) Patent Application Publication
(10) Pub. No.: US 2017/0156313 A1
(43) Pub. Date: Jun. 8, 2017

(54) PACKAGING FOR A MEDICAMENT USED FOR INNOVATIVE THERAPY

(71) Applicant: LABORATOIRE FRANCAIS DU FRACTIONNEMENT ET DES BIOTECHNOLOGIES, Les Ulis (FR)

(72) Inventors: Benoit HERNANDEZ, Paris (FR); Sebastien PRINGUET, Saint Germain Les Arpajon (FR)

(21) Appl. No.: 15/325,730

(22) PCT Filed: Jul. 17, 2015
(86) PCT No.: PCT/FR2015/051977

§ 371 Date: Jan. 12, 2017

(30) Foreign Application Priority Data
Jul. 18, 2014 (FR) 14 56935

(51) Int. Cl. A01N 1/02; A61J 1/14; B65D 81/38; A61J 1/10; A61K 39/00; B65B 7/00

(52) U.S. Cl. CPC A01N 1/0273; A61K 39/00; B65B 7/00; B65D 81/38; A61J 1/10; A61J 1/1468

(57) ABSTRACT

Disclosed is a product including: a) at least one substance selected from recombinant nucleic acids, single cells and single tissues, the compound being contained in a primary container; and b) a secondary container containing the primary container, the secondary container consisting of an insulating material. Also disclosed is a method for transporting a substance selected from recombinant nucleic acids, single cells and single tissues, which uses the product.

The present invention relates to a product comprising:
a) at least one substance chosen from recombinant nucleic acids, isolated cells and isolated tissues, said substance being present in a primary container; and
b) a secondary container, in which the primary container is present, said secondary container consisting of insulating material.

Biological substances, in particular innovative therapy medicaments, generally consist of living materials or materials resulting from living beings, such as nucleic acids, isolated cells or isolated tissues. Such substances are relatively delicate and require specific preservation and storage conditions in order to maintain their activity.

The usefulness of these substances is often impeded by logistical problems and in particular problems of conditions of transportation. For example, stem cells, such as cord blood cells, once collected, are commonly cryopreserved, in particular in cell libraries, and, in case of need, can be transported to hospitals. This cryopreservation process, according to which the cells or the tissues are preserved by cooling to temperatures typically of approximately −196° C., has certain risks. For example, the cells can be damaged by the freezing or during the reheating to ambient temperature. These risks are particularly serious for the maintenance of the activity of the biological substance as said substance has to retain its integrity. In addition, the cells and the isolated tissues used have to be viable in order to be effective.

The transportation of these biological substances thus has to be carried out over a period of time which is as short as possible. Specifically, deliveries during the night over dry ice or under liquid nitrogen constitute the norm and additional care has to be taken in order to control the temperatures throughout the journey. Nevertheless, this practice does not eliminate the risks. In addition, the transportation used is often expensive and not very practical over a long distance.

There thus exists a need to have available products and processes for the transportation of biological substances which are practical and reliable and which ensure maintenance of the integrity and of the viability of the biological substances concerned.

The present invention makes it possible to respond to this need. This is because the product according to the invention and the transportation process using this product provide monitoring and good conditions for the preservation of the biological substances over time. This is because, as demonstrated in the examples, transportation can be carried out over a period of time of 48 h while maintaining the set temperature, i.e. 2-8° C. or 34-38° C.

The present invention thus relates to a product comprising:
a) at least one substance chosen from recombinant nucleic acids, isolated cells and isolated tissues, said substance being present in a primary container; and
b) a secondary container, in which the primary container is present, said secondary container consisting of an insulating material.

The present invention also relates to a process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product according to the invention; and
iii) closing the secondary container obtained in stage ii).

The product and the transportation process according to the present invention relate to biological substances, which can be described as biopsy or innovative therapy medicaments.

These substances are chosen in particular from:
gene therapy medicaments;
somatic cell therapy medicaments;
medicaments resulting from cell or tissue engineering; and
combined innovative therapy medicaments.

Gene therapy medicaments generally consist of recombinant nucleic acids incorporated in appropriate tools (of the type of vectors, plasmids, viral particles and the like).

The other abovementioned medicaments typically consist of isolated cells or tissues. Thus, the substance is preferably chosen from recombinant nucleic acids, isolated mesenchymal stem cells, isolated hematopoietic stem cells and immunocompetent cells.

Hematopoietic stem cells can be used in allogenic or autologous fashion.

Immunocompetent cells are chosen in particular from allogenic T lymphocytes, NK cells, cytotoxic T lymphocytes, tumor-infiltrating lymphocytes (TILs) and antigen-sensitized dendritic cells, in particular when the antigen is a tumor antigen.

Some cell types can also be used, such as fetal neurons in the case of neurodegenerative diseases, islets of Langerhans or hepatocytes in replacement of organ grafts, or else fibroblasts, keratinocytes, chondrocytes, endothelial cells or myoblasts in the case of reparative medicine.

The product according to the invention thus comprises at least one substance a) chosen from recombinant nucleic acids, isolated cells and isolated tissues. This substance, also known as “biological substance” in the present invention, is present in a primary container. The primary container is for its part present in the secondary container b), said secondary container consisting of an insulating material.

Preferably, the insulating material constituting the secondary container is a polymer, preferably a plastic polymer. Preferably, the polymer is chosen from polypropylene, polytetrafluoroethylene (PTFE), polyethylene, polycarbonate, polyethylene terephthalate (PET), polyvinyl chloride (PVC), polyvinylidene fluoride and their mixtures. More preferably, the insulating material constituting the secondary container is polypropylene.

Preferably, the secondary container is a box or a case.

Preferably, the primary container is chosen from a bag, a tube, a bottle, a vial, a syringe, a microtube and a well plate. It is this which is in contact with the substance.

The biological substance can be formulated in a biologically acceptable medium within the primary container. “Biologically acceptable medium” is understood to mean a medium compatible with said substance. Such a medium is in particular a cell culture medium, for example comprising serum, such as human serum.

According to a preferred alternative form of the invention, the product according to the invention is present in a tertiary container comprising a heat regulation unit. Said tertiary container preferably consists of a heat regulation unit and of thermal insulation packs. The product according to the invention can also be contained in any other type of tertiary packaging, for example a polystyrene packaging or a packaging of vacuum insulating panel (VIP) type.

Preferably, the product according to the invention comprises a temperature probe, preferably a wire temperature probe. This probe is preferably in the secondary container in contact with the primary container. Such a probe makes it possible to monitor the temperature of the biological substance to the nearest point, without opening the containers concerned.

Preferably, the product according to the invention is coated with a layer of material resistant to X-rays, preferably a sheet of lead. Such a material resistant to X-rays makes it possible to protect the biological substance, in particular during transportation by plane.

Preferably, the primary, secondary and if appropriate tertiary containers of the product according to the invention are leaktight. They thus each ensure the insulation of the biological substance and in particular the transportation thereof in a reliable fashion.

Another subject matter of the invention is a process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product according to the invention; and
iii) closing the secondary container obtained in stage ii).

The transportation process according to the invention makes it possible to optimize the handling and the temperature maintenance and to preserve the integrity of the product (thus of the biological substance) with regard to external attacks which can be caused by cooling or heating means.

The transportation of the product according to the invention is possible with temperature maintenance for 72 h.

Preferably, the process makes possible the transportation of the substance in a stable fashion for a period of time of at least 24 h, preferably for at least 48 h.

Transportation “in a stable fashion” is understood to mean that the transportation is carried out without substantial deterioration of the biological substance, said biological substance preserving its physical integrity and its functionality. “Without substantial deterioration” means destruction of the substance of at most 5% by weight.

The invention will now be exemplified using the examples which follow, which are not limiting.

### EXAMPLE 1: TRANSPORTATION OF VACCINE

A biological substance, in the case in point a vaccine, is transported under different conditions.

The maintenance of the temperature is evaluated during the duration of the transportation.

Determination of the Time for the Fall in Temperature of a Product Stabilized at Ambient Temperature and Introduced into a Casing Equipped with a Temperature Regulating Unit, Everything being Placed in a Chamber Under the ST96A Profile

### 1-1 Tertiary Container (Casing)

Packaging: Casing R11

External dimensions: 394×310×521 mm

Working dimensions: 171×267×224 mm

Insulator: Polyurethane foam and VIP panels

Separators: a separator made of plastic fiber

### 1-2 Temperature Regulating Unit

Designation: Rigid Snowgam 10/05

Arrangement: individual

Number of packs: 1

Dimensions: 180×145×35 mm

Weight of the pack: 670 g

Designation: Rigid Snowgam 12/05

Arrangement: individual

Number of packs: 2

Dimensions: 230×180×35 mm

Weight of the pack: 890 g

Designation: TRU (Thermal Regulating Unit)

Arrangement: individual

Number: 1

Dimensions: 300×200×150 mm

Weight: 7.38 kg

### 2—Measurement of the Temperature

Thermobuttons used and positions:

Thermobutton number: THB 03 Location: Ambient temperature

Thermobutton number: THB 08 Location: Product

Thermobutton number: THB 10 Location: Casing interior

### 3—Test Conditions

Type of products: Empty syringe (primary container) placed in a cardboard packaging with dimensions of 43×23×133 mm.

The syringe is placed inside a polypropylene box with dimensions of 200×190×110 mm (secondary container). Kraft paper was placed around the vaccine in order to immobilize it.

### 4—Conditions of Loading

Temperature: +21 (±3° C.)

Arrangement of the Rigid Snowgam 10/05 pack: at the bottom at +5° C.

Arrangement of the Rigid Snowgam 12/05 packs: 1 on each long side at +5° C.

Arrangement of the TRU: on the top at −21° C.

Arrangement of the plastic fiber separator: between the TRU and the polypropylene box containing the product.

The R11 casing was charged with the different cold accumulators and also the TRU and the polypropylene box (with immobilization); everything being placed in the test chamber for 24 hours (first 24 hours of the profile). After these 24 hours, the casing was opened and the vaccine (stabilized at ambient temperature) was added to the load.

### ST96A Profile:

ST96A

| Temperature | Duration |
|---|---|
| +22° C. | 3 H |
| +28° C. | 4 H |
| +22° C. | 9 H |
| +28° C. | 8 H |
| +40° C. | 4 H |
| +28° C. | 3 H |
| +22° C. | 9 H |
| +25° C. | 8 H |
| +22° C. | 3 H |
| +28° C. | 4 H |
| +22° C. | 9 H |
| +28° C. | 8 H |
| +40° C. | 4 H |
| +28° C. | 3 H |
| +22° C. | 9 H |
| +25° C. | 8 H |

### Conclusion

In this configuration, the temperature of a vaccine introduced at ambient temperature into the casing equipped with 1 Rigid Snowgam 10/05, 2 Rigid Snowgam 12/05 and 1 TRU reaches 8° C. after testing for 30 minutes.

Subsequently, the casing is capable of maintaining the vaccine between +2° C. and +8° C. for at least 96 hours in this configuration.

### EXAMPLE 2: TEST OF VALIDATION OF THE INSULATING PROPERTIES OF THE SECONDARY CONTAINER WHEN EMPTY

The aim is to demonstrate that, at a very low temperature, the polypropylene packaging (i.e. secondary container) makes it possible to reduce the thermal impact which might potentially detrimentally affect the integrity of a substance chosen from recombinant nucleic acids, isolated cells and isolated tissues.

The other objective is to demonstrate that, after transportation, when the secondary packaging made of polypropylene has returned to ambient temperature, the latter slows down the rise in temperature and thus makes possible a more secure handling for the substance.

The test is as follows:

A polypropylene packaging is placed in a freezer <−60° C. for 10 h. A probe A (probe of Sensitech TempTale 4 Dry Ice type, which makes possible measurements from +60° C. to −110° C.) is placed inside the packaging and another B (of the same type) is placed outside the packaging.

This test is carried out in a room having a temperature regulated at 23° C.

The test began on Apr. 17, 2014 at 18 h 51 and came to an end on Apr. 18, 2014 at 10 h 07.

### Result:

### The Probe A was Placed Inside the Packaging:

### Fall in Temperature:

Maximum value: 22.5° C. at Apr. 17, 2014 18:51

Target freezing value: <−60.0° C. at Apr. 17, 2014 18:59

Time for falling from the maximum value (ambient temperature) to the target freezing value (ambient temperature of the freezer): 8 minutes

### Rise in Temperature:

Minimum value: −77.5° C. at Apr. 18, 2014 09:51

Value above 0° C. (2.6° C.) at Apr. 18, 2014 09:57

Maximum value: 22.9° C. at Apr. 18, 2014 10:07

Time for rising from the minimum value (ambient temperature of the freezer) to the maximum value (ambient temperature): 16 minutes

Time for rising from the minimum value (ambient temperature of the freezer) to the first value above 0° C.: 6 minutes

### The Probe B was Placed Outside the Packaging:

### Fall in Temperature:

Maximum value: 22.5° C. at Apr. 17, 2014 18:51

Target freezing value: <−60.0° C. at Apr. 17, 2014 18:55

Time for falling from the maximum value (ambient temperature) to the target freezing value (ambient temperature of the freezer): 4 minutes

### Rise in Temperature:

Minimum value: −77.9° C. at Apr. 18, 2014 09:51

Value above 0° C. (10.6° C.) at Apr. 18, 2014 09:54

Maximum value: 22.7° C. at Apr. 18, 2014 10:00

Time for rising from the minimum value (ambient temperature of the freezer) to the maximum value (ambient temperature): 9 minutes

Time for rising from the minimum value (ambient temperature of the freezer) to the first value above 0° C.: 3 minutes.

### Conclusion:

It is found that the polypropylene packaging (secondary container) provides a more gentle fall in temperature and also a slower rise in temperature. Specifically, all the parameters are on average multiplied by two.

### EXAMPLE 3: TEST OF VALIDATION OF THE INSULATING PROPERTIES OF THE SECONDARY CONTAINER

Two 50 ml Duran Schott flasks each filled with 25 ml of water are placed in a cold chamber in order to take the temperature.

The first flask is subsequently placed in an isothermal transportation bag and surrounded with two gel packs frozen beforehand to −80° C.

The second flask is first of all placed in the polypropylene packaging (secondary container) before being placed in an isothermal transportation bag and surrounded with two gel packs frozen beforehand to −80° C.

Each isothermal bag is equipped with a wire probe, one of which will be placed inside the isothermal packaging. The probes used are probes of Sensitech TempTale 4 Dry Ice type which make possible measurements from +60° C. to −110° C.

The isothermal packaging is placed beforehand in the cold chamber in order to also be at temperature.

The aim is to demonstrate that the polypropylene packaging makes it possible to reduce the impact of the excess freezing caused by gel packs which have been frozen too much placed in a refrigerated truck, which might potentially detrimentally affect the integrity of a substance chosen from recombinant nucleic acids, isolated cells and isolated tissues, or also distort the temperature reading during transportation.

### Results:

The test began on 22/04/14 at 16 h 36 and came to an end on 23/04/14 at 9 h 33.

A much smaller fall in the temperature measured by the probe inside the polypropylene packaging is observed, in comparison with the temperature measured by the probe placed directly in the middle of the two gel packs.

The difference in fall in temperature is approximately −20° C.

This test allows it to be concluded that a polypropylene packaging placed between two gel packs frozen beforehand to −80° C. for 10 h and allowed to defrost for approximately 4 h inside an isothermal transportation bag makes it possible to provide maintenance of the temperature between +2° C. and +8° C. for approximately 13 h.

### Conclusion:

It is found that the polypropylene packaging provides a much smaller fall in temperature than the same product not packaged in a polypropylene packaging.

### EXAMPLE 4: TEST OF VALIDATION OF THE INSULATING PROPERTIES OF THE SECONDARY CONTAINER

This protocol is targeted at testing the progression and the conditions under which the withdrawn samples are transported. A buffer solution or water is used to simulate the tissues or cells withdrawn.

The objective of this report is to describe the transfer of tissues/cells between a hospital and the site of production of the innovative therapy medicament. With the aim of mimicking this transfer of withdrawn tissues/cells, two tests were carried out:
one using a buffer of PBS formulation, on 6 May 2014, over a temperature of +34/+38° C.;
the other using water, on 17 Sep. 2014, over a temperature of +34/+38° C.

On each occasion, the objective is to guarantee the integrity of the flask during the transportation, from the removal up to the delivery, at a controlled temperature.

### Materials Used:

### Buffer or Water:

The formulation buffer mimicking the withdrawn sample (first test) was prepared beforehand on the hospital site. A 50 ml Sarstedt tube containing this buffer was thus prepared and placed in an incubator at +37° C. on May 6, 14 at 10 h 00.

| | Sarstedt tube |
|---|---|
| Name of the product | PBS |
| Description | Formulation buffer |
| Dimension | d. 2 cm h. 10 cm |
| Volume | 50 ml |
| Labeling | NA |

The water mimicking the withdrawn sample (second test) was prepared beforehand on the hospital site. A 50 ml Sarstedt tube containing this water was thus prepared and placed in an incubator at +37° C. of the Nantes University Hospital Center (UHC) on Sep. 17, 2014 at 10 h 00.

| | Sarstedt tube |
|---|---|
| Description | Water |
| Dimension | d. 2 cm h. 10 cm |
| Volume | 50 ml |
| Labeling | NA |

### External Packaging:

The external packaging is composed of a casing with packs.

This packaging was prepared in order to maintain a temperature of +34/+38° C.

### Packaging Protocol:

The Sarstedt tube containing the PBS or the water is placed in a 95 kPa plastic bag with a capacity of 11 (secondary packaging), then in a polypropylene box (200×190×110 mm) and then in the external packaging (394×310×521 mm).

### Probes:

Two probes were used for this transportation test:
a probe incorporated in the packaging, programmed at +34/+38° C.;
a Thermobutton probe programmed with a measurement interval of 3 min for recording. Temperature +34/+38° C.

The results of the first test are as follows:

| | | | | Internal |
|---|---|---|---|---|
| | Time | | | temperature |
| Actions | elapsed | Date | Hour | [+34° C.; +38° C.] |
| Starting probes | 00:00:00 | 5 May 2014 | 09:00:00 | 23.2° C. |
| Installation of the | 01:51:00 | 5 May 2014 | 10:51:00 | 21.9° C. |
| accumulators | | | | |
| Rise in temperature | 02:06:00 | 5 May 2014 | 11:06:00 | 34.1° C. |
| Departure package | 02:51:00 | 5 May 2014 | 11:51:00 | 37.6° C. |
| Opening: Handling | 05:24:00 | 5 May 2014 | 14:24:00 | 36.2° C. |
| at point A | | | | |
| Arrival package on | 1 Day | 6 May 2014 | 13:15:00 | 35.5° C. |
| the hospital site | 04:15:00 | | | |
| Opening: Addition | 1 Day | 6 May 2014 | 13:27:00 | 35.1° C. |
| of the withdrawn | 04:27:00 | | | |
| sample | | | | |
| Departure package + | 1 Day | 6 May 2014 | 13:36:00 | 34.5° C. |
| withdrawn sample | 04:36:00 | | | |
| Arrival package + | 2 Days | 7 May 2014 | 12:15:00 | 35.4° C. |
| withdrawn sample A | 03:15:00 | | | |
| Opening: Recovery | 2 Days | 7 May 2014 | 14:45:00 | 35.4° C. |
| of the withdrawn | 05:45:00 | | | |
| sample | | | | |
| Temperature < than | 5 Days | 10 May 2014 | 23:33:00 | 33.9° C. |
| 34° C. | 14:33:00 | | | |

| Index | Internal Temperature (° C.) |
|---|---|
| Specification | [+34.0; +38.0]° C. |
| Maximum | 37.8° C. |
| Minimum | 21.9° C. |
| Mean during the transportation | 35.5° C. |
| [05 May 2014 10:51- | |
| 07 May 2014 14:45] | |

The tube arrived under good conditions and adhering to the set points for the temperatures during the transportation.

The results of the second test were as follows:

| | | | | Internal |
|---|---|---|---|---|
| | Time | | | temperature |
| Actions | elapsed | Date | Hour | [+34° C.; +38° C.] |
| Starting probes | 00:00:00 | 16 Sep. 2014 | 11:18 | 36.1° C. |
| Departure package: | 00:06:00 | 16 Sep. 2014 | 11:24 | 36.3° C. |
| Sofrigam to Ulis | | | | |
| Opening: Handling | 03:27:00 | 16 Sep. 2014 | 14:45 | 35.5° C. |
| CfC Ulis | | | | |
| Arrival package on | 21:24:00 | 17 Sep. 2014 | 08:42 | 35.5° C. |
| the site of the | | | | |
| Nantes UHC | | | | |
| Opening: Addition | 1 Day | 17 Sep. 2014 | 11:51 | 34.8° C. |
| withdrawn sample | 00:33:00 | | | |
| Nantes UHC | | | | |
| Departure package + | 1 Day | 17 Sep. 2014 | 11:54 | 34.8° C. |
| withdrawn sample | 00:36:00 | | | |
| Nantes UHC | | | | |
| Arrival packaging + | 2 Days | 18 Sep. 2014 | 12:36 | 35.4° C. |
| withdrawn sample | 01:18:00 | | | |
| CfC Ulis | | | | |
| Opening: Recovery | 2 Days | 18 Sep. 2014 | 16:18 | 30.1° C. |
| withdrawn sample | 04:54:00 | | | |
| CfC Ulis | | | | |
| Rise in temperature | 2 Days | 18 Sep. 2014 | 16:33 | 34.2° C. |
| after closing | 05:15:00 | | | |
| Temperature < than | 6 Days | 22 Sep. 2014 | 13:33 | 32.5° C. |
| 34° C. | 02:15:00 | | | |

| Index | Internal Temperature (° C.) |
|---|---|
| Specification | [+34.0; +38.0]° C. |
| Maximum | 37.8° C. |
| Minimum | 24.9° C. |
| Mean during the transportation | 35.5° C. |
| [17 Sep. 2014 11:51- | |
| 18 Sep. 2014 16:18] | |

The tube arrived under good conditions and adhering to the set points for the temperatures during the transportation.

In conclusion, these transportation operations are in accordance: the material transported was maintained at a good temperature and for at least 48 h.

1-10. (canceled)

11. A product comprising:
a) at least one substance chosen from recombinant nucleic acids, myoblasts, isolated mesenchymal stem cells, isolated hematopoietic stem cells and immunocompetent cells, said substance being present in a primary container; and
b) a secondary container, in which the primary container present, said secondary container consisting of an insulating material, and said product being present in a tertiary container comprising a heat regulation unit.

12. The product as claimed in claim 11, wherein the insulating material constituting the secondary container is a polymer chosen from polypropylene, polytetrafluoroethylene, polyethylene, polycarbonate, polyethylene terephthalate, polyvinyl chloride, polyvinylidene fluoride and their mixtures.

13. The product as claimed in claim 11, wherein the primary container is chosen from a bag, a tube, a bottle, a vial, a syringe, a microtube and a well plate.

14. The product as claimed in claim 11, further comprising a temperature probee.

15. The product as claimed in claim 11, the product being coated with a layer of material resistant to X-rays.

16. The product as claimed in claim 11, wherein the primary and secondary containers are leaktight.

17. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 11; and
iii) closing the secondary container obtained in stage ii), said product being present in a tertiary container comprising a heat regulation unit.

18. The product of claim 14, wherein the temperature probe is a wire temperature probe.

19. The product of claim 15, wherein the layer of material resistant to X-rays is a sheet of lead.

20. The product as claimed in claim 16, wherein the tertiary container is leaktight.

21. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 12; and
iii) closing the secondary container obtained in stage ii), said product being present in a tertiary container comprising a heat regulation unit.

22. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 13; and
iii) closing the secondary container obtained in stage ii), said product being present in a tertiary container comprising a heat regulation unit.

23. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 14; and
iii) closing the secondary container obtained in stage ii), said product being present in a tertiary container comprising a heat regulation unit.

24. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 15; and
iii) closing the secondary container obtained in stage ii), said product being present in a tertiary container comprising a heat regulation unit.

25. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 16; and
iii) closing the secondary container obtained in stage ii), said product being present in a tertiary container comprising a heat regulation unit.

26. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 18; and
iii) closing the secondary container obtained in stage ii), said product being present in a tertiary container comprising a heat regulation unit.

27. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 19; and
iii) closing the secondary container obtained in stage ii), said product being present in a tertiary container comprising a heat regulation unit.

28. A process for the transportation of a substance chosen from recombinant nucleic acids, isolated cells and isolated tissues comprising the following stages:
i) placing said substance in a primary container;
ii) placing said primary container obtained in stage i) in a secondary container, and thus obtaining a product as claimed in claim 20; and
iii) closing the secondary container obtained in stage ii), said product being present in a tertiary container comprising a heat regulation unit.
